(19) United States
(12) Patent Application Publication
(10) Pub. No.: US 2006/0058650 A1
(43) Pub. Date: Mar. 16, 2006

(54) System and method for determinationof fetal movement

(76) Inventor: Reuven Sharony, Kfar Saba (IL)

Correspondence Address: NATH & ASSOCIATES, 112 South West Street, Alexandria, VA, 22314, US

(21) Appl. No.: 10/539,087

(22) PCT Filed: Dec. 15, 2003
(86) PCT No.: PCT/IL03/01069

§ 371 Date: Jun. 15, 2005

Related U.S. Application Data
(60) Provisional application No. 60/433,682, filed on Dec. 16, 2002.

(51) Int. Cl. A61B 8/00

(52) U.S. Cl. 600/437

(57) ABSTRACT

According to one aspect, the invention concerns a method for measuring motion of a fetus or of a region of a fetus within an uterus in a time sequence of images, each image including said fetus or region thereof and at least a portion of the uterus, the uterus comprising a lumen, the method comprising, for one or more images in the sequence (a) determining a first parameter indicative of movement of the fetus or region thereof; (b) determining a second parameter indicative of movement of at least a portion of the uterus; and (c). According to another aspect, the invention concerns for measuring motion of a fetus or of a region of a fetus within an uterus, the system comprising (a) an imaging device for obtaining a time sequence of images, each image including said fetus or region thereof and at least a portion of the uterus, the uterus comprising a lumen; (b) a processing utility for processing said time sequence of images so as to obtain at least one first parameter indicative of movement of the fetus or region thereof and at least one second parameter indicative of movement of at least a portion of the uterus; and (c) a display utility for displaying said at least one first and said at least one second parameter. According to yet other aspects, the invention concerns a computer program being readable by a computer for executing the method of the invention and a machine-readable memory tangibly embodying a program of instructions executable by the machine for executing the method of the invention.

### FIELD OF THE INVENTION

This invention relates to the field of non-invasive diagnostic method and system, and in particular to a method and system for objective determination of fetal movement by the use of imaging device.

### PRIOR ART

The following is a list of related art considered to be relevant as background of the invention. Appearance of a document in this list should not be construed as implying that the document is relevant to the patentability of the invention:

1. U.S. Pat. No. 5,111,823;

2. U.S. Pat. No. 5,178,147;

3. U.S. Pat. No. 5,782,766;

4. U.S. Pat. No. 5,605,155

5. International Patent Publication No. WO 03/003,899

### BACKGROUND OF THE INVENTION

The term SONAR refers to Sound Navigation and Ranging. As early as 1822, Daniel Colladen, a Swiss physicist, had successfully used an underwater bell to accurately determine the speed of sound in the waters of Lake Geneva. Later on, “The Theory of Sound” in which the fundamental physics of sound vibrations (waves), transmission and refraction was established. It was then first described that sound wave is a mathematical equation, forming the basis of any theoretical work in acoustics. Since then, echo-sounding techniques have extremely developed, to be used in a wide range of applications, including, inter alia, Obstetric Ultrasound. Several developments in the echo-sounding techniques are described in U.S. Pat. Nos. 5,111,823, 5,178,147, 5,782,766, and 5,605,155.

Obstetric Ultrasound is the use of ultrasound scans in pregnancy. Since its introduction in the late 1950's ultrasonography has become a very useful diagnostic tool in Obstetrics.

Currently used equipments in Obstetrics are known as real-time scanners, with which a continuous picture of the moving fetus can be depicted on a monitor screen. Very high frequency sound waves of between 3.5 to 7.0 megahertz (i.e. 3.5 to 7 million cycles per second), are generally used for this purpose. Sound wavers are emitted from a transducer placed in contact with a maternal abdomen and repetitive arrays of ultrasound beams scan the fetus in thin slices and are reflected back onto the same transducer. The information obtained from different reflections is recomposed back into a picture (typically, B-scan sonogram or ultrasonogram image) on a monitor screen (a sonogram, or ultrasonogram).

Movements such as fetal heart beat and malformations in the fetus are assessed and measurements are made on the images displayed on the screen. Such measurements form the cornerstone in the assessment of gestational age, size and growth in the fetus.

The uses of ultrasonography include diagnosis and confirmation of early pregnancy, determination of viability of a fetus in the presence of vaginal bleeding in early pregnancy, determination of gestational age and assessment of fetal size, diagnosis of fetal malformation, placental localization, multiple pregnancies, hydramnios and oligohydramnios, etc.

Ultrasonic evaluation of fetal movement is also routinely performed. An average number of fetal movements per time period are determined. A fetus that does not exhibit a typical pattern of movements in a given time is considered to be in distress, and if movement cannot be solicited, immediate measures are called for.

A method and apparatus for fetal movement diagnostics is described in WO 03/003,899. According to this publication, the movement of a fetus or an organ thereof is associated with a change in time in the intensity of pixels of an ultrasonic image including the fetus and the change.

### SUMMARY OF THE INVENTION

There is a need in the art to provide a system and method for objective, reproducible and automatic diagnostic of fetal movement. In the following description, the term “movement” is used interchangeably with the term “motion” and refers to change of place or position of a selected region within an image, in a time sequence of images, in comparison to the placement or position of the said region in a preceding image within the sequence. The present invention exploits the fact that an image comprises a plurality of pixels and by measuring changes in the intensity (increase or decrease in brightness) of a pixel compared to a corresponding pixel in a previous image, movement may be identified and determined.

The present invention further facilitates the determination of real (true) fetal movement. The determination of real fetal movement is achieved by taking into consideration movement originating from shifts in the placement of the transducer forming part of an imaging device which momentarily distort the image, movement of amniotic fluid surrounding the fetus, causing the fetus to drift within the amniotic sac, etc.

Thus, according to one aspect of the present invention there is provided a method for measuring movement of a fetus or of a region of a fetus within an uterus in a time sequence of images, each image including said fetus or region thereof and at least a portion of the uterus, the uterus comprising a lumen, the method comprising, for one or more images in the sequence:
(a) determining a first parameter indicative of movement of the fetus or region thereof;
(b) determining a second parameter indicative of movement of at least a portion of the uterus; and
(c) determining whether the difference between the first parameter and second parameter exceeds a predetermined value, a difference exceeding the predetermined value being indicative of movement of the fetus or region thereof relative to movement of the at least portion of the uterus.

The time sequence of images can be obtained by any conventional imaging device known in the art. According to one embodiment, the time sequence of images is obtained and recorded by an ultrasound device.

The method of the invention may be carried in real time, i.e. as the sequence of images are obtained from the imaging device (i.e. directly from the video output of the imaging device) or by retrieval of data from a storage utility or pre-recorded video cassette.

According to the present invention several steps for calibration and noise reduction may be performed on the time sequence of images.

According to one embodiment, regions of the images containing irrelevant information (i.e., textual and graphical elements generated by the imaging device are deleted from the images.

According to another embodiment, primary and secondary noise reductions on the images are performed.

According to yet another embodiment, histogram equalization of each image within the sequence of images is performed.

For the purpose of determining real movement of the fetus it is required according to the present invention that movement in areas outside the lumen be distinguished from movement of the fetus within the uterus. To this end, the method of the invention comprises the step of marking the contour of the lumen of the uterus in a first image of the sequence of images. According to a preferred embodiment, the contour of the lumen as marked in the first image is then tracked from image to image throughout the time sequence of images recorded. The tracking of the contour of the lumen is achieved by a special algorithm capable of locating in each image the new location of points representative of the contour of the lumen.

According to the invention, motion in sequence is detected. To this end, in each image after the first image in the sequence, clusters of pixels showing movement (identified by change in intensity of a pixel) are detected and the size of each cluster is determined. Preferably, clusters having a size not exceeding a pre-defined threshold are discarded. Clusters located within the contour of the lumen and clusters located outside the contour of the lumen are distinctively marked (e.g. clusters within the uterus are marked with a color different from clusters outside the contour of the lumen).

According to one embodiment of the invention, in each image after the first image, a center of gravity of the clusters within the uterus, and a center of gravity of the clusters outside the contour of the lumen are determined. The term “center of gravity” as used herein denotes the point at which the total clusters of pixels which showed movement relative to a preceding image (either within the contour of the lumen or outside the contour of the lumen) may be considered as concentrated.

According to a preferred embodiment, the locations of the two centers of gravity are tracked from image to image and in each image, for each of the two centers of gravity, a displacement vector is determined. The term “displacement vector” as used herein denotes the difference between the location of a center of gravity in a selected image and the location of the center of gravity in the following image and may be represented as an arrow drawn from a center of gravity in a selected image to the center of gravity in the following image.

According to a further embodiment, the method of the invention comprises a step of calculating for each image after a first image in the sequence of images, a motion density outside the contour of lumen of the uterus, and a motion density within the uterus. The term “motion density” as used herein interchangeably with the term “movement density” denotes the number of pixels in a marked region (either within the lumen or outside the contour of the lumen) displaying motion divided by the total number of pixels in that region. According to a preferred embodiment of the invention, images having a high motion density outside the contour (i.e., the motion density outside the contour exceeds a pre-defined threshold) and/or images having a low motion density inside the contour of the lumen (i.e., the motion density within the contour do not exceed a pre-defined threshold) are identified and discarded.

Thus, according to a preferred embodiment of the invention subsequences of images having a low motion density outside the contour of the lumen and having a high motion density within the lumen are identified and processed. Further according to a preferred embodiment, images in which the vector difference of the two displacement vectors (outside the contour and within the contour) has a magnitude exceeding a predetermined threshold are identified. According to the invention, these identified images are images displaying real (true) motion of the fetus.

Parameters of detected real motion of the fetus are thus generated by the method of the invention. Such parameters may be, for example, the identified motion densities, the displacement vectors, the velocity of movements, shift in centers of gravity, rotations etc.

According to one embodiment of the invention, the parameters calculated are compared with pre-defined parameters indicative of a normal fetal movement. From the comparison a medical practitioner may objectively judge the fetal well-being.

As may be appreciated by those versed in the art of obstetrics abnormal fetal movement (hyper or hypoactive as compared to a predetermined average normal scale) may correlate with a medical disorder, which, in the absence of an alternative diagnostic tool, will be detected only after birth. For example prenatal fixation especially in the upper limbs and neck may suggest Arthrogryposis Multiplex Congenita (AMC) which is a syndrome complex characterized by multiple joint contractures without other serious congenital abnormalities and with relatively normal intelligence. In addition, lack (as compared to a predefined normal value) of movement in a limb or a palm may suggest Hemiparesis (weakness on one side of the body) or Trisomy.

Further, lack of reduced movements associated with swallowing may suggest Familial Dysautonomy, Hypoxic Ischemic Encephalopathy, or In Utero brain injury/Hemorrhage.

In the absence of an efficient diagnostic tool, such as that suggested by the present invention, some of the disorders, will be detected three months or more after birth, such as cerebral palsy (CP), mild hypoxic ischemic encephalopathy, mental retardation (the latter associated with reduced yawning of the fetus) and Down Syndrome.

Thus, according to the present invention it is possible, at any stage of the prenatal life of a fetus, to determine movement of a specific limb or organ of the fetus and thereby diagnose a disorder, such as those described above. It is a unique feature of the present invention which enables the focusing on the movement of a specific limb or organ from which a medical condition of the fetus may be concluded, long before delivery is due and allow the medical practitioners to determine whether to interfere (e.g. stop) with the pregnancy.

According to a second aspect of the invention, there is provided a system for measuring motion of a fetus or of a region of a fetus within a uterus, the system comprising:
(a) imaging device for obtaining a time sequence of images, each image including said fetus or region thereof and at least a portion of the uterus, the uterus comprising a lumen;
(b) a processing utility for processing said time sequence of images so as to obtain at least one first parameter indicative of movement of the fetus or region thereof and at least one second parameter indicative of movement of at least a portion of the uterus;
(c) a display utility for displaying said at least one first and said at least one second parameter.

According to one embodiment within this aspect of the invention, the processing utility comprises means for determining whether the difference between the first parameter and the second parameter exceeds a predetermined value, a difference exceeding the predetermined value being indicative of motion of the fetus or region thereof relative to motion of the at least portion of the uterus.

The system of the invention preferably comprises means for marking in an image a contour of the lumen of the uterus in the image. Such means may be in the form of an edge detection algorithm as is known in the art of image processing, or may be performed manually by using a user input interface such as a computer mouse or a keyboard.

According to one embodiment, the processing utility comprises a tracking utility for tracking the contour of the lumen from image to image.

The processing utility according to the invention comprises means for determining movement outside the contour of the lumen and movement within the lumen.

Preferably, the processing utility comprises means for detecting in each image after a first image in the time sequence of images, clusters of pixels displaying a change in brightness in comparison to brightness of a corresponding pixel in a previous image.

Yet preferably, the processing utility is equipped with means for distinctively marking clusters located within the contour of the lumen and clusters located outside the contour of the lumen.

According to one embodiment of the invention, the processing utility comprises means for determining a center of gravity of clusters within the contour of the lumen and a center of gravity of clusters outside the contour of the lumen, wherein for each image in the sequence of images, said center of gravity of clusters within the contour of the lumen and a center of gravity of clusters outside the contour of the lumen form a pair of centers of gravity.

The processing utility preferably also comprises means for tracking from image to image in said time sequence of images the location of the center of gravity of clusters within the contour of the lumen and determining a displacement vector of said center of gravity in each image.

According to this embodiment, the processing utility also comprises means for tracking from image to image in said time sequence of images the location of the center of gravity of clusters outside the contour of the lumen and determining a displacement vector of said center of gravity in each image.

According to another embodiment, the processing utility comprises means for calculating for each image after said first image, motion density outside the contour of the lumen and motion density within the lumen. According to this embodiment, it is preferable that the processing utility is equipped with means for discarding images having a motion density outside the contour of the lumen which exceeds a pre-defined threshold and discarding images having a motion density within the lumen which does not exceed a pre-defined threshold and for identifying subsequences of images having simultaneously a low motion density outside the contour and a high motion density within the contour.

According to a further embodiment of the invention, the processing utility comprises means for determining for each pair of displacement vectors the angular difference or the magnitude ratio and identifying images in which said values exceed a predetermined threshold.

The system of the invention may comprise a display utility for receiving from the processing utility one or more parameters indicative of motion and displaying the same. Alternatively or in addition, the system of the invention may comprise an output utility for receiving from the processing utility one or more parameters indicative of motion and outputting the same.

The system of the invention may also comprise a storage utility for receiving and storing the time sequence of images recorded by said imaging device and for recording one or more parameters determined by said processing utility.

The system of the invention may also comprise a user input interface for assisting in the performance in the different functionalities of the system. For example, the user input interface may be applied for recording data corresponding to the specific subject being diagnosed (e.g. age of female subject, age of fetus, calculated weight, etc.). The user input interface may also be utilized for providing the processing with parameters necessary for the performance of the processing session, such as the marking of the contour of the lumen.

It will be understood that the system according to the invention may include a suitably programmed computer. Likewise, the invention contemplates a computer program being readable by a computer for executing the method of the invention. The invention further contemplates a machine-readable memory tangibly embodying a program of instructions executable by the machine for executing the method of the invention. According to the invention, it is preferable that the machine-readable memory for executing the method of the invention may be used in existing computer systems as an “add-on” product.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a schematic illustration of a system according to one embodiment of the present invention for determining movement of a fetus.

FIG. 2 is a schematic block diagram of the method steps performed according to one embodiment of the present invention.

### DETAILED DESCRIPTION OF THE INVENTION

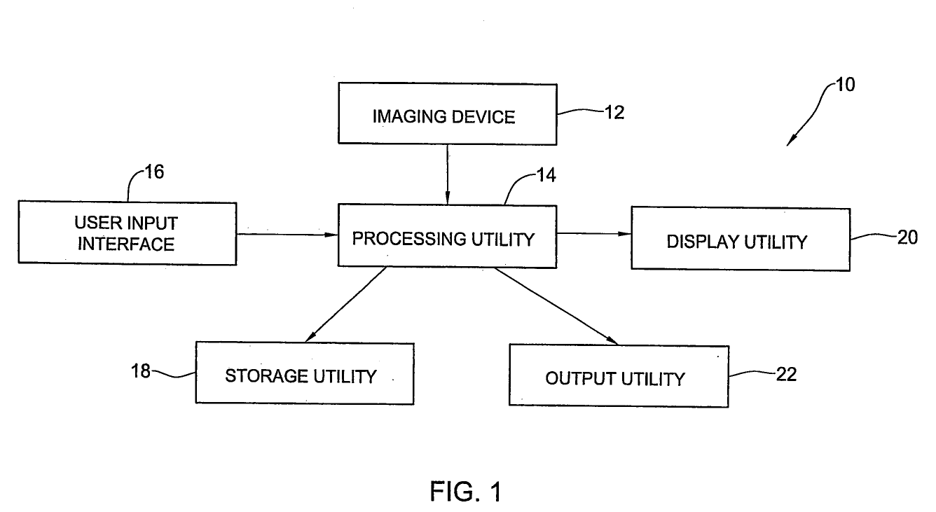

Referring to FIG. 1, there is illustrated a measurement system 10 suitable to be used in the method of the present invention. Measurement system 10 comprises such main constructional parts as an imaging device 12 and a processing utility 14 connectable to each other via wires or wirelessly (by using IR, RF or acoustic signaling). Processing utility 14 may be a separate computer device, part of a subjects personal digital assistant (PDA, e.g., PALM) a cellphone, or integral processing utility of the imaging device 12.

The imaging device 12 can be any known real-time scanner or developed in the future in the filed of medicine. Examples of commercially available and commonly used real-time scanners include, without being limited thereto: 2D, 3D and 4D ultrasound machines or combinations thereof, such as Voluson 530 or is Voluson 730 (Medison America Inc. Cypres, Calif., USA); Doppler ultrasound; and others known to the practitioner and commercially available.

Imaging device 12 is preferably equipped with a user input interface 16 designed to enable input from a user (technician or medical practitioner having expertise in operating an ultrasonic device) to initiate a measurement session and to enter data relevant to the measurement session including time/day, calculated age of the fetus,.and any other parameters relevant for the purpose of the image processing and data retrieval. In addition, imaging device 12 may also receive input from storage utility 18 including archived clinical data. Data associated with parameters of one or more fetal body parts is recorded by imaging device 12 and then processed by processing utility 14 and may also be stored on the storage utility 18. Digital recording of images is not limited to a specific resolution or speed of recording; though obviously preferred parameters of resolution and recording speed will give optimal results.

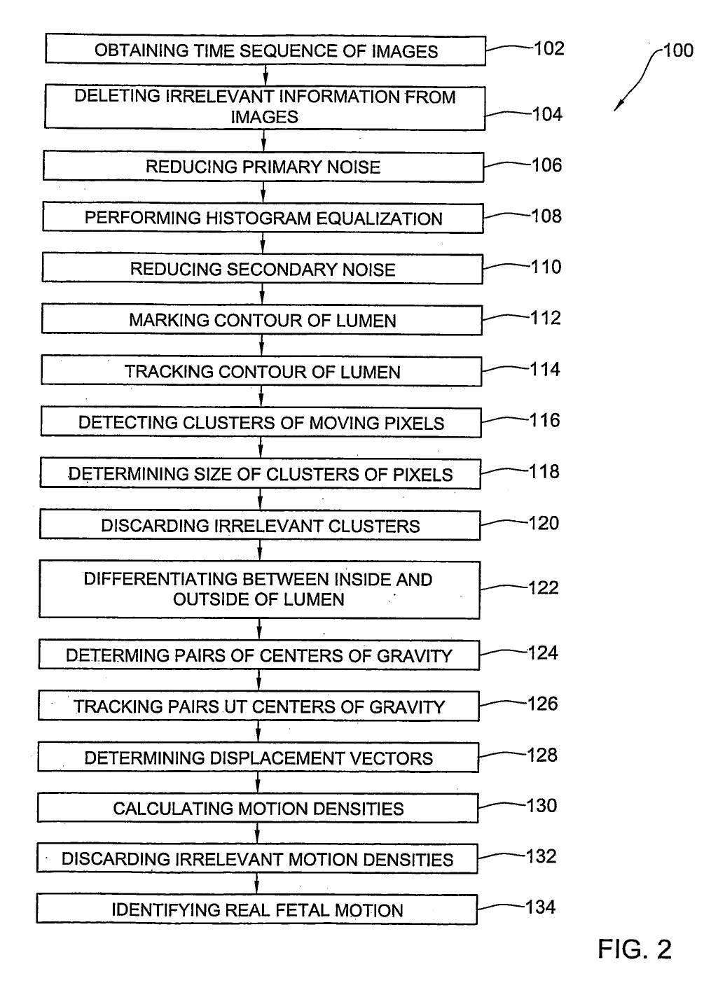

The output of imaging device 12, is preferably in a pixel or voxel form, and is supplied into the processing utility 14 which is described hereinafter in greater detail with reference to FIG. 2.

A display utility 20, which may be a visual (e.g. color or gray hues display) optionally combined with an audible message is preferably associated with the imaging device 12, the processing utility 14, and the user input interface 16 and provides the user with a sequence of images indicative of the fetal movement as well as of the various functionalities of the processing utility 14. The processing utility 14 processes data received from imaging device 12 and generates a visual graphical display on the display utility 20 which is indicative of the fetal movement (e.g. a graph, a histogram, a typographical map etc.).

The results of the processing may also be converted to an audible message which is rendered audible by a speaker associated with display utility 20.

Further, alternatively or in addition, the processing utility 14 preferably provides an output to an output utility 22, such as a printer (which is associated with display utility 20) outputting the data, e.g. in a graphical form.

The processing utility 14 is preprogrammed to collect measured data from the imaging device and/or from the storage utility 18 and carry out data analysis consisting of determining true movements of a fetus or of a selected organ or region of the fetus, as a function of certain parameters selected by the user and entered into processor utility 20 via user input interface 16.

Processing utility 14 may be proximal to the imaging device 12 or at a remote site and connected to the imaging device 14 and the display/output utilities 18, 22 by means of a computer network (e.g. the Internet, Intranet) or by means of a communication link.

Reference is now made to FIG. 2 which is a schematic block diagram 100 of the main steps carried out by the processing utility 14. In step 102 a time sequence of images is obtained using an imaging device 12. The images may be processed either in real time, directly from the video output of the imaging device 12, after storage of the images in the storage utility 18, or from a pre-recorded video cassette.

In step 104, regions of the images containing irrelevant information (i.e., textual and graphical elements generated by the imaging device 12) are deleted from the images. Deletion of these regions may be performed manually using a user input interface such as a computer mouse, or may be done automatically by the processing utility 14.

In step 106, primary noise reduction on the images is performed. As well appreciated, sources of noise that may reduce the quality of the image and cause the detection of false movement of the fetus include periodic noise due to imperfections in the system (e.g. in a VCR system) and speckle noise generated by the imaging device. Primary noise reduction may be performed by batch filtering, thresholding, local low pass filtering or by any other means known in the field of image is processing and which enable the automatic detection, filtration and removal spectral components of periodic or acoustic noise (ultrasonic speckles).

A process of histogram equalization is then performed (step 108). A histogram of each image in the sequence is generated and modified in order to compensate for variation in brightness and contrast from image to image in the sequence. Secondary noise reduction is then performed by local low pass filtering of the images (step 110).

In step 112, the contour of the lumen of the uterus in a first image of the sequence is marked in the image. The contour may be marked either manually using a user input interface such as a computer mouse or a keyboard. Alternatively, the contour may be detected using an edge detection algorithm as is known in the art of image processing.

In step 114, the contour of the lumen is tracked from image to image. A plurality of points on the contour in the first image is tracked in the sequence of images, and the location of each of the points associated with the contour of the lumen is determined in each image. A point located in an image that causes an abrupt change in the curvature of the contour of the lumen displayed in the image is discarded, and replaced with a new point whose location is determined by interpolation of neighboring points located on the contour, so as to generate a smooth contour. In this manner a closed contour is created in each image that can be tracked in the sequence.

In step 116, motion in the sequence is detected. Particularly, in each image, after the first image, clusters of pixels showing movement are detected. To this end, pixels showing movement are those pixels displaying a change in brightness (either an increase or decrease) in comparison to the brightness of the corresponding pixel in the previous image.

In step 118, the size (in pixels) of each cluster is determined, and in step 120, clusters having a size not exceeding a pre-defined threshold are discarded.

In each image, clusters located within the contour of the lumen and clusters located outside the contour of the lumen are distinctively marked (step 122).

In each image after the first image, the center of gravity of the clusters within the contour of the lumen, and the center of gravity of the clusters outside the contour of the lumen are determined (step 124). Then, the locations of the two (the “pair”) centers of gravity are tracked from image to image to image (step 126). In each image, for each of the two centers of gravity, a displacement vector is drawn from the location of the center of gravity in the previous image to the center of gravity in the present image (step 128).

In addition, for each image after the first image, a motion density outside the lumen of the uterus, and a motion density within the uterus, are calculated (step 130). The motion density is defined as the number of pixels in the region (either within the contour of the lumen or outside the contour) displaying motion divided by the total number of pixels in the region.

In step 132, images having a high motion density outside the contour (i.e., the motion density outside the contour exceeds a pre-defined threshold) are identified. Such apparent motions are typically artifacts associated with changes between images induced by movements of the transducer, movements of the mother, drifts of the fetus within the amniotic sac, etc. In addition, images having a low motion density inside the contour of the lumen (i.e., the motion density within the contour do not exceed a pre-defined threshold) are discarded. Low motion density inside the contour of the lumen typically result from noise of the imaging device.

Subsequences of images having a low motion density outside the contour of the lumen and having a high motion density within the contour of the lumen are identified. The determination of parameters indicative of motion outside the contour of the lumen and within the lumen is necessary for performing the method of the invention. Only images in which the angular difference or magnitude ratio of the two displacement vectors (outside the contour and within the contour) having a value exceeding a predetermined threshold are identified (step 134). These identified images are images displaying real (true) motion of the fetus.

Parameters of detected real motion may be introduced into the images; displayed separately on the display utility 20 or outputted on the output utility 22. Such parameters may be, for example, the identified motion densities, the displacement vectors, the velocity of movements, shift in centers of gravity, rotations etc. The parameters may be presented texturally or in a graphical form, for example as a histogram; a topographical map etc.

It should well be appreciated by those versed in the art that while the above specific embodiment movement of the entire lumen is detected; the method of the invention is also applicable for detection of movement of a specific region within the uterus. Thus, according to yet another embodiment of the invention, a specific limb or organ of the fetus may be selected and tracked in the sequence of images, and a motion density of the limb or organ calculated instead of or in addition to the motion density of the entire lumen of the uterus.

While only a selected embodiment have been described in detail herein, it is readily understood that many modifications and variations of the present invention are possible within the spirit, concept and scope of the invention as set out in the appended claims.

1. A method for measuring motion of a fetus or of a region of a fetus within an uterus in a time sequence of images, each image including said fetus or region thereof and at least a portion of the uterus, the uterus comprising a lumen, the method comprising, for one or more images in the sequence:
(a) determining a first parameter indicative of movement of the fetus or region thereof;
(b) determining a second parameter indicative of movement of at least a portion of the uterus; and
(c) determining whether the difference between the first parameter and second parameter exceeds a predetermined value, a difference exceeding the predetermined value being indicative of motion of the fetus or region thereof relative to motion of the at least portion of the uterus.

2. The method of claim 1 comprising a step of marking in an image a contour of the lumen of the uterus in the image.

3. The method of claim 2, comprising a step of tracking the contour of the lumen from image to image.

4. The method of claim 2, comprising a step of determining movement within the contour of the lumen, said movement being identified as movement of the fetus or region thereof.

5. The method of claim 2, comprising a step of determining movement outside the contour of the lumen, said movement being identified as movement of the uterus.

6. The method of claim 2, comprising a step of detecting in each image after a first image in the time sequence of images, clusters of pixels displaying a change in brightness in comparison to brightness of a corresponding pixel in a previous image.

7. The method of claim 6, comprising a step of discarding clusters of pixels having a size not exceeding a pre-defined threshold.

8. The method of claim 6, comprising the step of distinctively marking clusters located within the contour of the lumen and clusters located outside the contour of the lumen.

9. The method of claim 6, comprising the step of determining a center of gravity of clusters within the contour of the lumen and a center of gravity of clusters outside the contour of the lumen, wherein for each image, said center of gravity of clusters within the contour of the lumen and a center of gravity of clusters outside the contour of the lumen form a pair of centers of gravity.

10. The method of claim 9, comprising the step of tracking from image to image in said time sequence of images location of the center of gravity of clusters within the contour of the lumen and determining a displacement vector of the center of gravity in each image.

11. The method of claim 9, comprising the step of tracking from image to image in said time sequence of images the location of the center of gravity of clusters outside the contour of the lumen and determining a displacement vector of the center of gravity in each image.

12. The method of claim 11, comprising the step of calculating for each image after said first image, motion density outside the contour of the lumen and motion density within the lumen.

13. The method of claim 12, comprising the step of discarding images having a motion density outside the contour of the lumen which exceeds a pre-defined threshold and discarding image(s) having a motion density within the lumen which does not exceed a pre-defined threshold.

14. The method of claim 12, comprising the step of identifying subsequences of images having simultaneously a low motion density outside the contour and a high motion density within the contour.

15. The method of claim 10, comprising the step of determining in each image an angular difference or magnitude ratio between the displacement vector outside the lumen and the displacement vector within the uterus, and identifying images in which said angular difference or magnitude ratio has a value exceeding a predetermined threshold.

16. The method of claim 1, comprising the step of displaying one or more parameters indicative of motion.

17. The method of claim 16, wherein said parameter is selected from motion density, displacement vector, shift, rotation, velocity.

18. The method of claim 1, wherein said fetus or region thereof comprise the entire area within the lumen, the entire fetus, or a limb or organ of the fetus.

19. A system for measuring motion of a fetus or of a region of a fetus within an uterus, the system comprising
(a) imaging device for obtaining a time sequence of images, each image including said fetus or region thereof and at least a portion of the uterus, the uterus comprising a lumen;
(b) a processing utility for processing said time sequence of images so as to obtain at least one first parameter indicative of movement of the fetus or region thereof and at least one second parameter indicative of movement of at least a portion of the uterus;
(c) display utility for displaying said at least one first and said at least one second parameter.

20. The system of claim 19, wherein said processing utility comprises means for determining whether the difference between the first parameter and the second parameter exceeds a predetermined value, a difference exceeding the predetermined value being indicative of motion of the fetus or region thereof relative to motion of the at least portion of the uterus.

21. The system of claim 19, comprising means for marking in an image a contour of the lumen of the uterus in the image.

22. The system of claim 21, wherein said processing utility comprises a tracking utility for tracking the contour of the lumen from image to image.

23. The system of claim 21, wherein said processing utility comprises means for determining movement outside the contour of the lumen and movement within the lumen.

24. The system of claim 23, wherein said processing utility comprises means for detecting in each image after a first image in the time sequence of images, clusters of pixels displaying a change in brightness in comparison to brightness of a corresponding pixel in a previous image.

25. The system of claim 24, wherein said processing utility comprises means for distinctively marking clusters located within the contour of the lumen and clusters located outside the contour of the lumen.

26. The system of claim 25, wherein said processing utility comprises means for determining a center of gravity of clusters within the contour of the lumen and a center of gravity of clusters outside the contour of the lumen, wherein for each image in the sequence of images, said center of gravity of clusters within the contour of the lumen and a center of gravity of clusters outside the contour of the lumen form a pair of centers of gravity.

27. The system of claim 26, wherein said processing utility comprises means for tracking from image to image in said time sequence of images the location of the center of gravity of clusters within the contour of the lumen and determining a displacement vector of the center of gravity in each image.

28. The system of claim 26, wherein said processing utility comprises means for tracking from image to image in said time sequence of images the location of the center of gravity of clusters outside the contour of the lumen and determining a displacement vector of the center of gravity in each image.

29. The system of claim 19, wherein said processing utility comprises means for calculating for each image after said first image, motion density outside the contour of the lumen and motion density within the lumen.

30. The system of claim 29, wherein said processing utility comprises means for discarding images having a motion density outside the contour of the lumen which exceeds a pre-defined threshold and discarding images having a motion density within the lumen which does not exceed a pre-defined threshold.

31. The system of claim 29, wherein said processing utility comprises means for identifying subsequences of images having simultaneously a low motion density outside the contour and a high motion density within the contour.

32. The system of claim 31, wherein said processing utility comprises means for determining in each image an angular difference or magnitude ratio between the displacement vector outside the lumen and the displacement vector within the uterus, and identifying images in which said angular difference or magnitude ratio has a value exceeding a predetermined threshold.

33. A program storage device readable by machine, tangibly embodying a program of instructions executable by the machine to perform method steps for measuring motion of a fetus or of a region of a fetus within an uterus in a time sequence of images, each image including said fetus or region thereof and at least a portion of the uterus, the uterus comprising a lumen, the method steps being defined by claim 1.

34. A computer program product comprising a computer useable medium having computer readable program code embodied therein for measuring motion of a fetus or of a region of a fetus within an uterus in a time sequence of images, each image including said fetus or region thereof and at least a portion of the uterus, the uterus comprising a lumen, the computer program product comprising:
computer readable program code for causing the computer to determine a first parameter indicative of movement of the fetus or region thereof;
computer readable program code for causing the computer to determine a second parameter indicative of movement of at least a portion of the uterus; and
computer readable program code for causing the computer to determine whether the difference between the first parameter and second parameter exceeds a predetermined value, a difference exceeding the predetermined value being indicative of motion of the fetus or region thereof relative to motion of the at least portion of the uterus.

35-38. (canceled)
